(19) United States
(12) Patent Application Publication
(10) Pub. No.: US 2020/0375263 A1
(43) Pub. Date: Dec. 3, 2020

(54) MINIATURE GASEOUS SUBSTANCE RELEASE DEVICE

(71) Applicant: Jou-Tzu CHEN, Taipei City (TW)

(72) Inventor: Jou-Tzu CHEN, Taipei City (TW)

(21) Appl. No.: 16/430,031

(22) Filed: Jun. 3, 2019

(51) Int. Cl. A24F 47/00; A61M 15/06; A61M 11/04; A61M 21/00; H05B 1/02

(52) U.S. Cl. CPC A24F 47/008; A61M 15/06; A61M 11/042; A61M 2021/0016; H05B 1/0297; A61M 2205/3673; A61M 21/00

(57) ABSTRACT

A miniature gaseous substance release device is described, which is a portable miniature unit capable of transforming and releasing gaseous substances by means of introducing an external power and a release device for enabling user sensing, having a strip-shaped housing mounted therein, by means of filling, with a generation room where oil gas transformation occurs and a remaining space thereof being an oil compartment. The generation room obtains a working agent from the oil compartment via a drawing element and excites an oil gas via an electrothermal device provided therein, and the oil gas statically or dynamically diffuses and is guided outwards along a release end under the action of pressure.

### BACKGROUND OF THE INVENTION

### a) Field of the Invention

The present invention provides a miniature gaseous substance release device and, more particularly, a portable unit capable of transforming and releasing gaseous substances by means of introducing an external power and a release device for enabling olfactory sensing or acting on organs in users.

### b) Description of the Prior Art

A device related to aroma diffusion is provided with a design enabling capillary diffusion or vaporization and diffusion by electrical heating according to the materials of a transformation agent. The device is electrically driven and has a main body consisted of a bottle capable of accommodating a liquid agent, and a circular heating element provided at an opening of the bottle, wherein the heating element has an inner hole for enabling a fiber bundle to pass through, an inner end of the fiber bundle includes an invading fluid and an outer end thereof diffuses towards ambient air. Although the device can use a direct current power supply, the structure thereof is inherently inconvenient to be carried around. Functionally speaking, the device can only be operated in a mode of vaporization by heating; the inner fragrance molecules or the included treatment ingredients are driven by using the heat energy. In addition, similar device designs are also common in the treatment of respiratory tract, in which the vaporized and diffused gaseous molecules are drawn from externally, and the overall structure is inconvenient for carrying around.

### SUMMARY OF THE INVENTION

It is a major object of the present invention to provide a miniature gaseous substance release device which is a portable miniature unit capable of transforming and releasing gaseous substances by introducing an external power and a release device for enabling effects on the respiratory organ by inhaling or olfactory sensing in a user, said device has a strip-shaped housing provided therein, by means of filling, with a generation room where oil gas transformation occurs and a remaining space thereof being an oil compartment; the generation room obtains a working agent from the oil compartment via a drawing element and excites an oil gas via an electrothermal device provided therein; the oil gas is guided outwards along a release end under the action of pressure, thereby enabling a use of gaseous sensing for the user.

A further object of the present invention is to provide a vertical trough which is an upright trough for the strip-shaped housing, in which a top cover and a bottom seal base are combined together along a longitudinal direction of the vertical trough, thus facilitating molding, assembly and production if an embodiment employs a plastic material therein.

A third object of the present invention is to provide a fitting space in the electrothermal device for enabling contacting and fitting over an outer surface of an exchange end provided on a drawing element.

A fourth object of the present invention is to provide a fitting space formed by an electrothermal coil for the electrothermal device for enabling fitting by the drawing element, wherein the electrothermal coil is formed by winding and is easy to process.

A fifth object of the present invention is to provide a thermal diffusion element which is indirectly fitted between the fitting space of the electrothermal device and the drawing element, wherein the thermal diffusion element is a material having high thermal conductivity, and diffuses a heat energy obtained from the fitting space to an inner surface.

A sixth object of the present invention is to enable electrical conduction, via a passive switch, between the electrothermal device and an electrical connection end, wherein passive operation is enabled by using the passive switch to operate, thus saving energy and preventing the electrothermal device from accumulating heat excessively between uses.

A seventh object of the present invention is to provide a release channel capable of isolating the oil compartment which is communicated with between a connection tube of an air collector and a perforation of the top cover, wherein the release channel is communicated with the generation room and has an outer end thereof combined with a release end where the perforation is located.

An eighth object of the present invention is to provide a joint portion in a position of an outer end of a relay terminal provided on the electrical connection end, wherein the joint portion is externally connected to a connection element for electrical transmission, and the connection element can be a plug-in element or an electricity transmission connection wire having a flexible body.

A ninth object of the present invention is to provide a diffusion mode for the gaseous substances, which can be static or dynamic.

### BRIEF DESCRIPTION OF THE DRAWINGS

FIG. 1 is an exploded view showing an assembly structure of a release device in accordance with the present invention.

FIG. 2 is a schematic view showing an assembly relationship of the release device in accordance with the present invention.

FIG. 3 is a front view showing an assembled structure of the release device in accordance with the present invention.

FIG. 4 is another front view showing an assembled structure of the release device in accordance with the present invention.

FIG. 5 is a schematic view showing an electrothermal device in accordance with an embodiment of the present invention.

FIG. 6 is a structural cross-sectional view showing a thermal diffusion element in accordance with the present invention.

FIG. 7 is a schematic view showing that the electrothermal device is provided with a passive switch in accordance with the present invention.

FIG. 8 is a schematic view showing an electrical connection end externally connected to a connection element in accordance with the present invention.

FIG. 9 is a schematic view showing that a seal cover is fitted over an outer surface of the oil compartment in accordance with the present invention.

### DETAILED DESCRIPTION OF THE PREFERRED EMBODIMENTS

The present invention provides a miniature gaseous substance release device, including a release device 100 which is portable and capable of transforming and releasing gaseous substances by introducing an external power and enabling olfactory sensing for a user, a strip-shaped housing 11 mounted therein, by means of filling, with a generation room 20 where oil gas transformation occurs, wherein the generation room 20 acquires a working agent from an oil compartment 10 and excites an oil gas by using an electrothermal device 30 provided therein, and the oil gas is guided outwards along a release channel 50 under the action of pressure, thereby enabling gaseous sensing for the user; in order to facilitate convenience in assembly and production, a vertical trough 110 provided in the strip-shaped housing 11 has a shape of a upright trough, and a top cover 80 and a bottom seal base 22 are combined together along a longitudinal direction of the vertical trough 110, thus facilitating molding if an embodiment employs a plastic material therein; a fitting space 300 is provided in the electrothermal device 30 for enabling contacting and fitting over an outer surface of an exchange end 42 provided on a drawing element 40, such that the drawing element 40 can be positioned; for convenience in production, the electrothermal device 30 can be an electrothermal coil 31, and the fitting space 300 formed by the electrothermal coil 31 enables fitting by the drawing element 40.

A thermal diffusion element 32 is indirectly fitted between the fitting space 300 provided in the electrothermal device 30 and the drawing element 40, wherein the thermal diffusion element 32 is a material having high thermal conductivity, and diffuses a heat energy obtained from the fitting space 300 to an inner surface for the heat energy to act on a surface of the exchange end 42 included therein. With regard to the mode of electrical control, a passive switch 75 can be provided between the electrothermal device 30 and an electrical connection end 70, wherein passive operation is enabled by using the passive switch 75 to operate, thus saving energy and preventing the electrothermal device 30 from being burnt due to excessive heat accumulation between uses. With regard to leakproofing, a release channel 50 capable of isolating the oil compartment 10 is penetratingly provided between a connection tube 211 of an air collector 21 and a perforation 81 of the top cover 80, wherein the release channel 50 is communicated with the generation room 20 and has an outer end thereof combined with a release end 120 where the perforation 81 is located.

In addition, a joint portion is provided in a position of an outer end of a relay terminal 71 provided on the electrical connection end 70, wherein the joint portion is externally connected to a connection element 90 for electrical transmission, and the connection element 90 can be a plug-in element or an electricity transmission connection wire having a flexible body.

The present invention provides a device which is miniature and portable, which facilitates generation of gaseous substances for use at any time upon having a mobile power source supplied thereto, and a diffusion mode thereof includes static diffusion and dynamic diffusion.

The release device 100 of the present invention is a portable and miniature device for providing sensory uses by enabling a thermal excitation effect via an external power so as to drive oil molecules to become ionized. A working power thereof is dependent on importing externally, and the external power can employ any modes of connection; further, a source of the external power can be a power source having USB (Universal Serial Bus) output, such as a mobile powerbank, 3C mobile products like mobile phones or a converted direct-current power source when used indoor. The release device 100 is convenient to be carried around and can be used personally at any time and anywhere; the release of the gaseous substances can be static diffusion or dynamic diffusion by drawing via an external force.

A used oil agent L can be substantially a substance having a treatment effect, such as a fragrance for conditioning nerves, or a medical agent for treating a respiratory organ by inhaling, a nicotine compound for helping quit smoking; the agents are selected from agents capable of diffusing molecules by low temperature thermal catalysis and can be oil-based or water-based synthetic agents. The example used in the explanation is an oil agent.

The detailed structure of the present invention and operating modes thereof are illustrated in the following drawings and explanations:

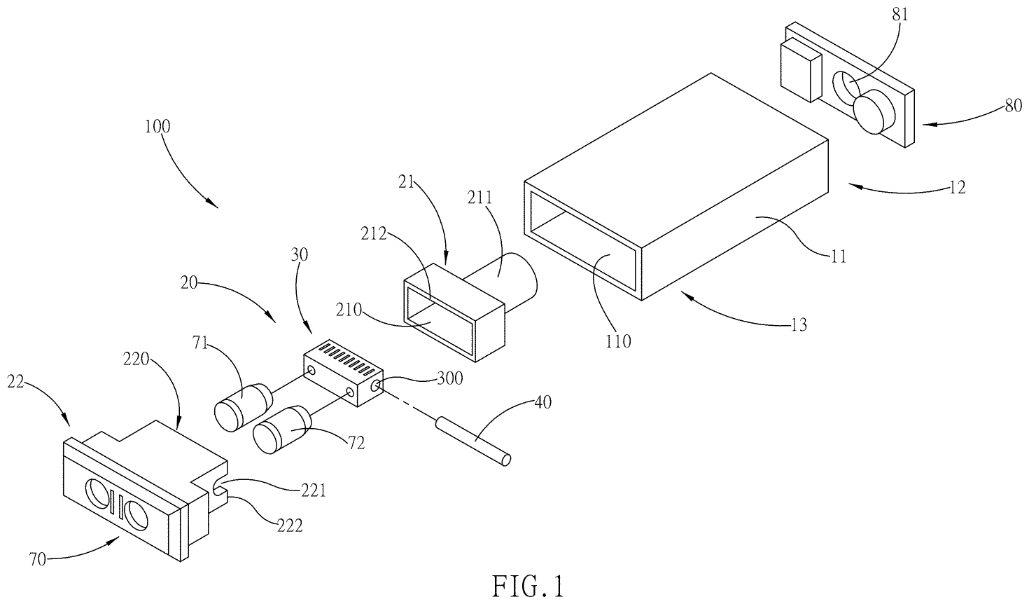

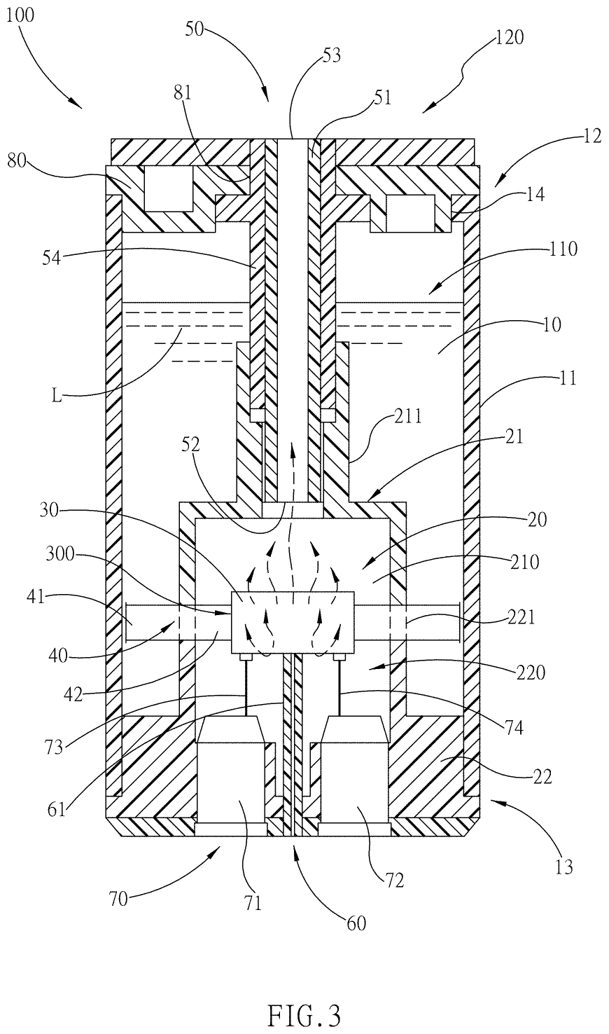

firstly, referring to FIG. 1, the release device 100 comprises a strip-shaped housing 11 which has a shape of a strip, wherein the strip-shaped housing 11 is penetratingly provided therein with a vertical trough 110 along a vertical direction thereof, and the vertical trough 110 is externally communicated with an upper-end opening 12 and a lower-end opening 13 of the strip-shaped housing 11; a top cover 80 provided with a perforation 81 and sealingly covers over the upper-end opening 12; a generation room 20 made up by an air collector 21 in combination with a bottom seal base 22 is provided in a space of the vertical trough 110, wherein the air collector 21 has an upper half compartment 210 provided therein and a matching port 212 provided on a lower end surface thereof, and the matching port 212 can be sealingly combined with a matching portion 222 provided on the bottom seal base 22; the bottom seal base 22 has a crossover port 221 concavely provided in the matching portion 222; after combination, the crossover port 221 and the upper half compartment 210 of the air collector 21 are integrated into the generation room 20 (refer to FIG. 3); an electrothermal device 30 is accommodated in a space within the upper half compartment 210 and a lower half compartment 220, and the electrothermal device 30 has electricity introduced thereto via a relay terminal 71 and a relay terminal 72 provided on an electrical connection end 70 configured on a bottom portion of the bottom seal base 22; the electrothermal device 30 is transversely provided with a fitting space 300, wherein the fitting space 300 enables fitting by a drawing element 40; the drawing element 40 can overpass the crossover port 221 of the bottom seal base 22; the lower-end opening 13 of the strip-shaped housing 11 can fit and seal the periphery of the bottom seal base 22; a connection tube 211 provided on the air collector 21 is communicated with the perforation 81.

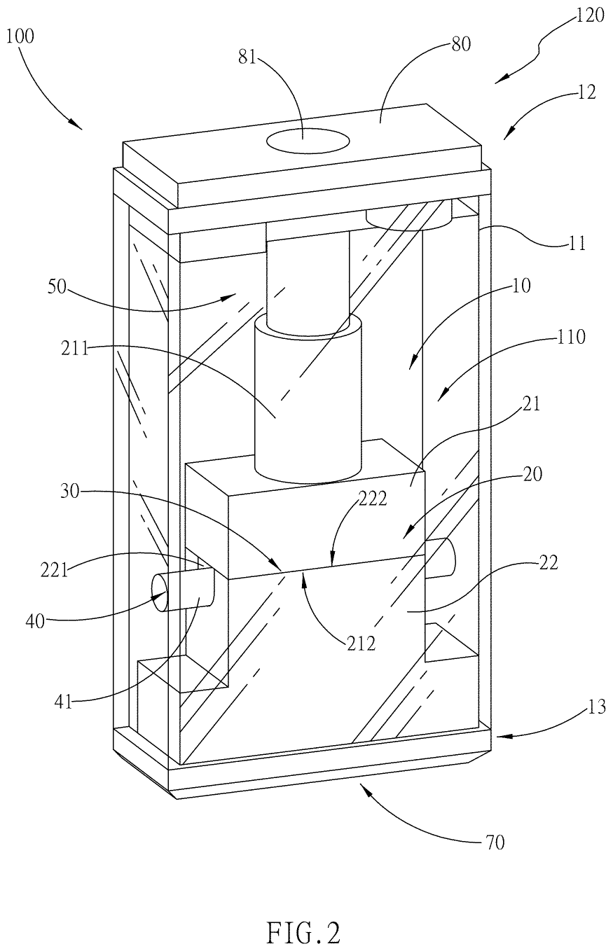

Referring to FIG. 2, after assembling the aforesaid structures, the release device 100 has the top cover 80 covered and sealed over the upper-end opening 12 of the strip-shaped housing 11 and the bottom seal base 22 sealed over the lower-end opening 13 of the strip-shaped housing 11, wherein an upper end of the bottom seal base 22 is combined with the air collector 21, such that the air collector 21 is communicated with the perforation 81 of the top cover 80 via the connection tube 211; the perforation 81 and the top cover 80 as a whole which comprise an overall surface of an upper end of the strip-shaped housing 11 are defined as a release end 120 of the release device 100.

The drawing element 40 has a drawing-in end 41 which exposes out of an outer surface of the bottom seal base 22; the matching port 212 of the air collector 21 and the matching portion 222 of the bottom seal base 22 are combined together in a watertight manner; the crossover port 221 of the bottom seal base 22 enables the drawing element 40 to overpass. An inner space resulted from the combination between the air collector 21 and the bottom seal base 22 forms the generation room 20, wherein the generation room 20 internally comprises the electrothermal device 30; electrical conduction is enabled for the electrothermal device 30 via the electrical connection end 70 at a bottom end. After accommodating the generation room 20, a remaining space of the vertical trough 110 in the strip-shaped housing 11 forms an oil compartment 10, wherein the oil compartment 10 is capable of accommodating an oil agent for transformation, and the exposed drawing-in end 41 intrudes into a space of the oil compartment 10.

Referring to FIG. 3 (in combination with FIG. 1), the release device 100 has a strip-shaped housing 11 internally penetrated by means of the vertical trough 110, in which the upper-end opening 12 is sealed by the top cover 80 and the top cover 80 has a supply hole 14, wherein the supply hole 14 is capable of enabling and disabling communication in the vertical trough 110; after assembly, the air collector 21 and the bottom seal base 22, which constitute the generation room 20, occupy the space in the vertical trough 110, and a remaining space of the vertical trough 110 is an oil compartment 10. The oil compartment 10 accommodates an oil agent L, and the drawing-in end 41 of the drawing element 40 is located in the space of the oil compartment 10 via the crossover port 221; an exchange end 42 is provided in a space of the generation room 20 and is fitted by a fitting space 300 of the electrothermal device 30.

The electrothermal device 30 is electrically connected to a relay terminal 71 and a relay terminal 72 of the electrical connection end 70 via a wire 73 and a wire 74, wherein the relay terminal 71 and the relay terminal 72 have outer ends exposed externally and are capable of conducting power from externally.

The connection tube 211 of the air collector 21 is communicated with the perforation 81 of the top cover 80, wherein a release channel 50 penetrates through the strip-shaped housing 11, a release outlet 53 of a provided lead-in tube 51 is communicated with the perforation 81 and covers over an outer surface of the top cover 80 together with an upper end of the strip-shaped housing 11, thereby collectively forming a release end 120; the release end 120 is capable of being suctioned and sipped by a user using mouth. The release channel 50 has a collection end 52 leading into the generation room 20; in order to achieve watertight combination, the release channel 50 can have an outer portion thereof fitted with a leakproof tube 54, in which the leakproof tube 54 can be an elastic material, so as to isolate the generation room 20 including the perforation 81 of the top cover 80 and the release channel 50 from the oil compartment 10.

The generation room 20 is formed by the upper half compartment 210 of the air collector 21 and the lower half compartment 220 of the bottom seal base 22 by means of matching the matching port 212 with the matching portion 222; the electrothermal device 30 is fitted into a space of the generation room 20, in which the electrothermal device 30 can be supported and positioned by the wire 73 and the wire 74, and the same is also held by the drawing element 40.

The oil compartment 10 supplies an oil agent L via the supply hole 14, and the oil agent L subsequently comes into contact with the drawing-in end 41 of the drawing element 40; the drawing-in end 41 has a capillary drawing action which draws the oil agent L into the exchange end 42, and then the oil agent L forms an oil film (not shown in the drawing) by means of an aggregation force of oil agent molecules on a surface of the exchange end 42; the oil film can be contacted with the fitting space 300 of the electrothermal device 30, and an electrothermal action is enabled by power introduction from the electrothermal device 30 via the electrical connection end 70, which generates heat molecules for undergoing heat exchange with the oil film of the exchange end 42, consequently promoting a swimming velocity of the oil agent molecules, generating gaseous molecules afterwards. In a gaseous transformation process resulted therefrom, changes in density causes molecule expansions, which produces different pressures in the space of the generation room 20, and the pressures are guided towards the perforation 81 from the connection tube 211 or pushed towards the release outlet 53 from the collection end 52, and then diffused out of the release end 120. The mode of diffusion is static diffusion without requiring an external force from outside of the release device 100, and the user can smell the odor molecules catalyzed by the generation room 20 up close.

A gas transformation amount diffused from the generation room 20 can be modified by changing the power of the electrothermal device 30 or a property of the filled oil agent L, thereby enabling adjustments of different levels in sensory sensing of the user.

With regard to a rate of odor molecule release diffused from the release end 120, a further embodiment of the present invention can be used to modify a rate of diffusion thereof by means of operations by external negative pressure and via compensation effect in atmospheric pressure, thereby enabling dynamic diffusion. The atmospheric pressure can be guided inwards from a balance channel 60 in a bottom portion of the bottom seal base 22, wherein the balance channel 60 is provided with a ventilation pipe 61 having an outer end thereof in contact with the ambient atmospheric pressure and an inner end thereof in communication with the generation room 20; when suctioning occurs at the release end 120 via an external force, such as a general air suctioning device or by mouth of a user, a negative pressure generated in the generation room 20 is guided by the balance channel 60 so as to balance the external atmospheric pressure, an airflow amount in a path of the release channel 50 is increased, and an increase in the airflow amount can elevate a rate of gaseous substance release transformed by the electrothermal device 30. The electrothermal device 30 can further have a power thereof increased so as to satisfy a requirement of heat energy for transformation. The supply hole 14 provided on the strip-shaped housing 11 which is located on the upper-end opening 12 can be movably sealed by an inner surface of the top cover 80; the top cover 80 can be movably opened and closed in relation to the supply hole 14; an outer surface of the bottom seal base 22 in the bottom portion of the strip-shaped housing 11 and an inner surface of the lower-end opening 13 are fixedly combined in a sealed manner, such as a fixed combination by adhesion or by ultrasonic fusion, or for the purpose of facilitating maintenance, the bottom seal base 22 and the lower-end opening 13 can be combined in a blocking manner, but the combination thereof must be watertight.

The oil compartment 10 accommodates the oil agent L, in which the oil compartment 10 is a sealed space; when the oil agent L is to be released towards a direction of the exchange end 42, atmospheric pressure must be filled into the oil compartment 10, so that the atmospheric pressure can be guided into the oil compartment 10 via the crossover port 221 by means of capillary action of the drawing element 40, and the oil agent L can be released towards the exchange end 42, wherein the crossover port 221 joined with the drawing-in end 41 can have a gap preserved therebetween, so that a fluid can be distributed over the outer surface of the exchange end 42 along the gap.

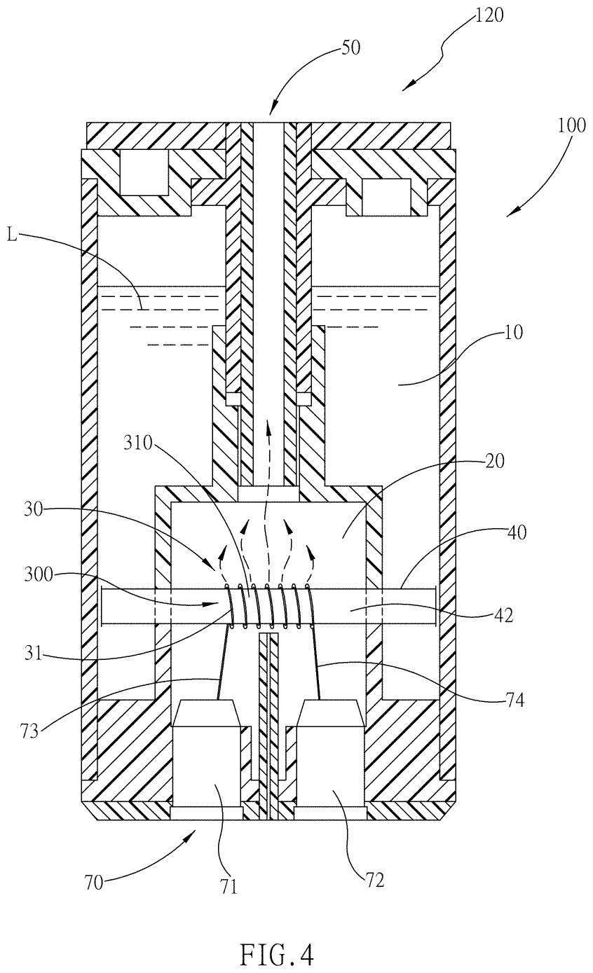

Referring to FIG. 4, for the purpose of facilitating production, the electrothermal device 30 provided in the generation room 20 in the release device 100 can be designed as an electrothermal coil 31, wherein the electrothermal coil 31 have two ends thereof respectively connected to the relay terminal 71 and the relay terminal 72 provided on the electrical connection end 70 via the wire 73 and the wire 74; the electrothermal coil 31 is formed by winding spirally, a spiral span 310 serving as a spiral pitch is present between every two coils, and the coil axially and internally form a fitting space 300, wherein the fitting space 300 enables the exchange end 42 of the drawing element 40 to pass through; a heat energy generated by the electrothermal device 30 acts on the surface of the exchange end 42 and enables heat exchange by contact, so as to heat the oil film of the oil agent L and excites the oil film to form gaseous oil gas molecules to be output via the release channel 50. The electrothermal coil 31 is made by means of a simpler manner, and adjustments of a power thereof can be determined by an external power.

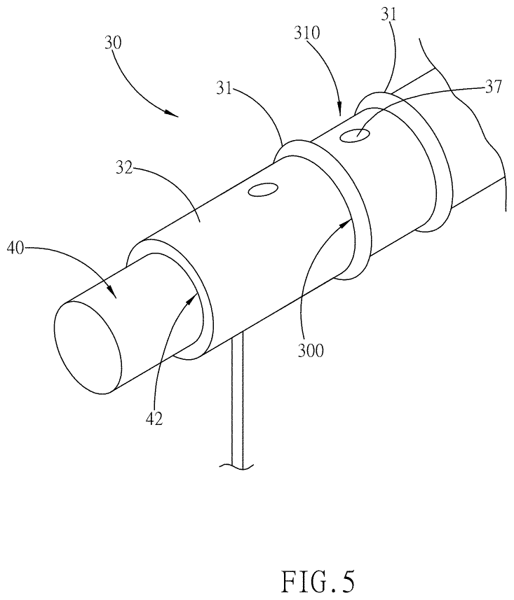

Referring to FIG. 5, a thermal diffusion element 32 is indirectly fitted between the fitting space 300 of the electrothermal device 30 and the exchange end 42 of the drawing element 40, wherein the thermal diffusion element 32 is made of a material having a high thermal conductivity coefficient, and an outer surface thereof receives a heat energy of the electrothermal device 30, and then the heat energy indirectly passes through a main body of the thermal diffusion element 32 via heat balance, thus bringing heat towards the surface of the exchange end 42 for heat exchange to occur.

The thermal diffusion element 32 can be a tubular body and has an inner circle for accommodating the exchange end 42; the thermal diffusion element 32 is transversely and openly provided with air holes 37 in a lengthwise range, so as to release pressure and smoke generated internally from the air holes 37.

The electrothermal device 30 is an electrothermal coil 31, and an air hole 37 is openly provided between the spiral spans 310 of the electrothermal coil 31, such that coils of the electrothermal coil 31 do not block off a path of the air hole 37, and the generated heat can be uniformly distributed between two air holes 37.

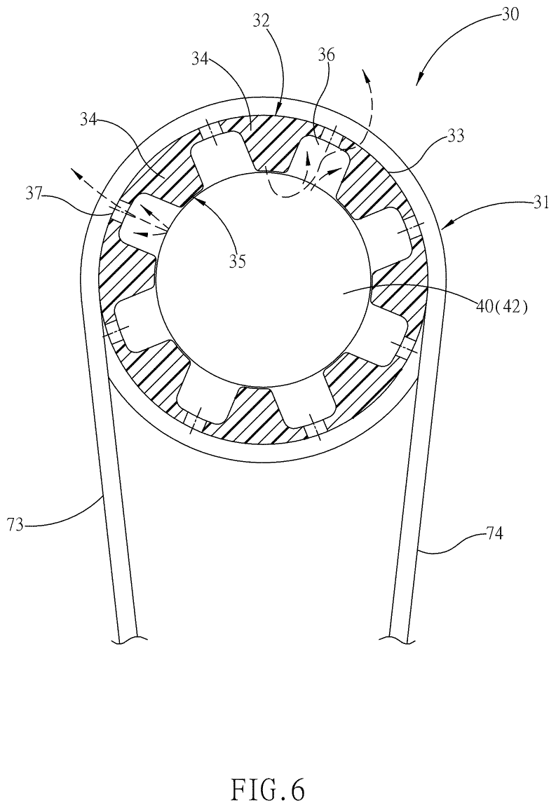

Referring to FIG. 6, an air slot 36 is concavely provided along a widthwise direction on an inner surface of the thermal diffusion element 32, in which a remaining inner gear tooth face 35 is contacted with the outer surface of the exchange end 42 of the drawing element 40; the air slot 36 provides a diffusion path for the smoke; the thermal diffusion element 32 has a thermal conductive surface 33 in contact with an electrothermal element, such as the electrothermal coil 31; a heat energy generated by the electrothermal coil 31 is guided towards an inner gear rack 34 via the thermal conductive surface 33 and is indirectly and uniformly conveyed to the inner gear tooth face 35, so as to heat the oil film on the surface of the exchange end 42 (not shown in the drawing); a smoke diffused from the surface can be diffused via the air slot 36, or via the air hole 37 openly provided between the air slot 36 and the thermal conductive surface 33 for vertical communication, so as to help the smoke in the air slot 36 diffuse outwardly. The position of the air hole 37 is indicated in FIG. 5, which is indirectly in the lengthwise range between the spiral spans 310.

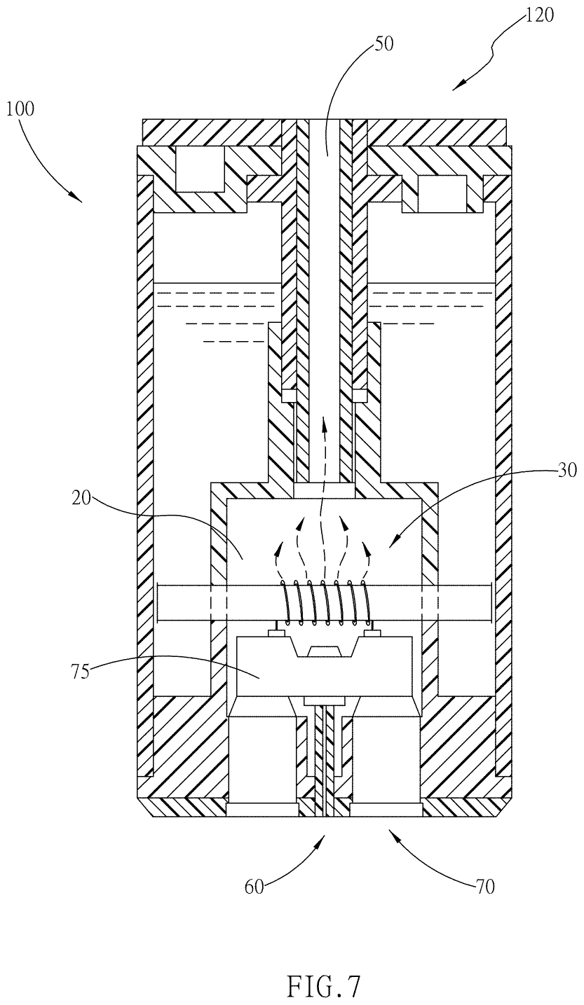

Referring to FIG. 7, an internal pressure of the generation room 20 is changed when the release device 100 is operated by an external force via the release end 120, and if a negative pressure is formed, a positive pressure is compensated by the balance channel 60; a time of operation of the electrothermal device 30 can be determined by a passive switch 75, in which the passive switch 75 is provided between the electrothermal device 30 and the electrical connection end 70, wherein the passive switch 75 is a unit operated under the action of gas pressure, and sensing in changes of airflow pressures can be achieved by detecting pressure changes in a path leading from the balance channel 60 towards the generation room 20 via the passive switch 75, which determines whether the passive switch 75 is operated and subsequently enables conduction of electricity between the electrical connection end 70 and the electrothermal device 30 or not; if a pressure of the generation room 20 is in equilibrium with the atmospheric pressure, the operation of the passive switch 75 is stopped so as to enable the electrothermal device 30 to be under the influence of an external pressure difference; if a negative pressure is present, the passive switch 75 drives the electrothermal device 30 to operate, so that power is only supplied to the electrothermal device 30 for performing heat exchanges during dynamic diffusion, thereby enabling the electrothermal device 30 to stop operations when not in use and saving energy.

The passive switch 75 can also be a thermal sensitive operation element; when an external force applied on the release end 120 brings about dynamic diffusion, a thermal sensitive effect thereof is used to sense changes in airflows and temperatures, such that changes in an electron amount generates an electrical current order for assigning an operation time to the electrothermal device 30.

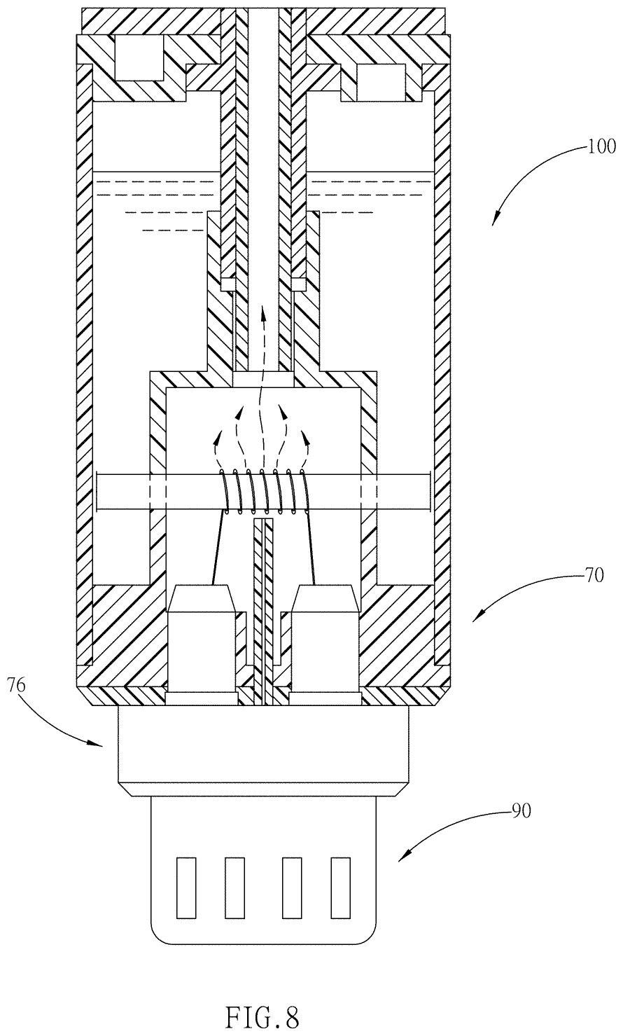

Referring to FIG. 8, the electrical connection end 70 provided in the release device 100 can have a joint portion 76 provided on an outer surface thereof, wherein the joint portion 76 can be externally connected to an element for conducting electricity, such as a plug, a socket or an extension cord, and the plug or the socket can be a connection element 90 having USB; the same can be further provided with an electrical cord for crossing over, wherein a body of the cord is flexible and deformable, and a deformed cord is capable of holding and securing the release device 100 at any angle and in any position.

The joint portion 76 is a magnetic structure capable of enabling insertions, engagements or fixing by mutual magnetic attractions; the structure has a general design and will not be described in details here, and mainly serves the purpose of enabling combination and electrical connection between the connection element 90 and the electrical connection end 70.

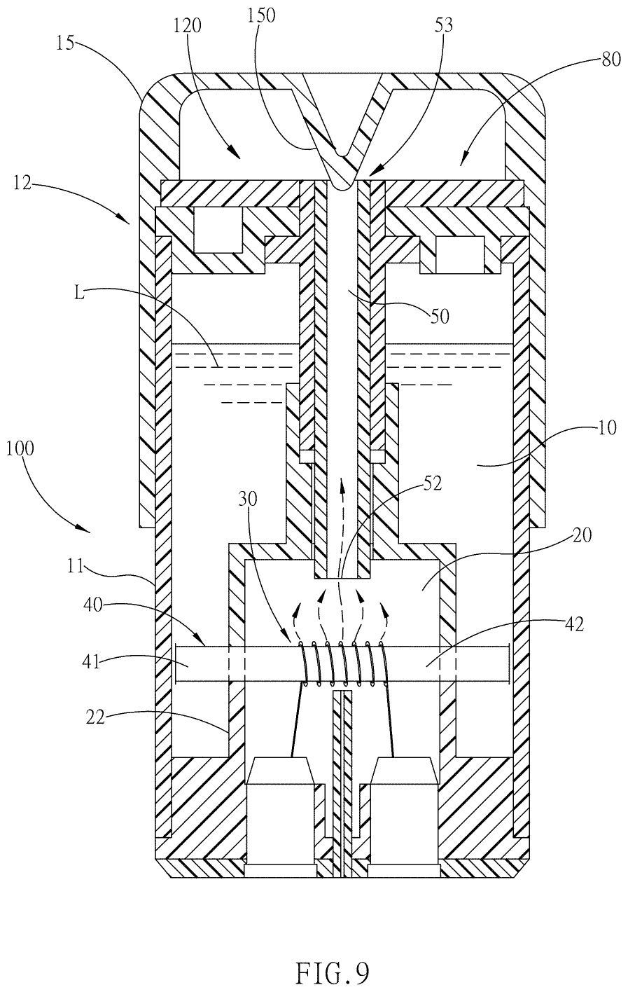

Referring to FIG. 9, the top cover 80 covers and seals over the bottom seal base 22 provided on the strip-shaped housing 11 of the release device 100, and the periphery of an upper half part thereof can be fitted with a seal cover 15, wherein the fitting of the seal cover 15 enables a sealed state as indicated in the drawing, thus helping prevent an oil agent from leaking out of the oil compartment 10.

The oil agent L is thermally driven and diffused as molecules by the electrothermal device 30 after entering into the exchange end 42 from the drawing-in end 41 of the drawing element 40; under a certain condition, when an input power of the electrothermal device 30 is insufficient for driving and diffusing an oil amount drawn by the exchange end 42, remaining oils become accumulated in the generation room 20; in addition, the capillary action of the exchange end 42 normally draws in the oil agent L from the oil compartment 10, such that oils become accumulated in a corner where the exchange end 42 is joined with an inner wall of the generation room 20 and form accumulated oils, and the accumulation is further increased due to viscosity of oil molecules, resulting in the oils becoming adhered to the inner surface of the generation room 20; subsequently, once the adhered oil film reaches a certain amount, the oils excessively accumulated in the generation room 20 will leak out of the release outlet 53 under the action of gravity when the release device 100 is placed at a certain angle.

In order to directly block off the oils, a stud 150 is provided at an exit of the release end 120, such as a position of the release outlet 53, so as to surely prevent leakage during storage.

The stud 150 can be integrally provided with the seal cover 15 and the two can be operated synchronously; when the seal cover 15 covers and seals the release end 120, the stud 150 also covers and seals the release outlet 53.

As described previously, the oils excessively accumulated in the generation room 20 will spread to the release channel 50 from the corner where the exchange end 42 is joined with the inner surface of the generation room 20; in order to proactively prevent the accumulated oils from leaking out of the release channel 50, the release outlet 53 provided in the release channel 50 is configured to be suspended in the generation room 20 in an intruding manner, such that the oils adhered to the inner surface of the generation room 20 are prevented from crossing the release outlet 53 regardless of what angle the release device 100 is placed at, thereby preventing the oils from leaking.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A miniature gaseous substance release device being a portable release device capable of transforming and releasing gaseous substances by introducing an external power and enabling olfactory sensing, comprising:
a strip-shaped housing having a vertical trough provided therein, wherein one end of the strip-shaped housing is an upper-end opening and another end thereof is a lower-end opening, and a front end and a rear end of the vertical trough are respectively communicated with the upper-end opening and the lower-end opening;
a top cover having a perforation provided thereon, wherein the top cover covers and seals over the upper-end opening, and outer surfaces of the top cover and the upper-end opening collectively form a release end;
a generation room entirely accommodated in the vertical trough by filling, wherein a remaining space resulted after filling the vertical trough is an oil compartment, a constitution of a main body of the generation room further comprises:
an air collector having therein an upper half compartment and a matching port at a lower-end opening end thereof, wherein the upper half compartment is communicated with the perforation via a connection tube;
a bottom seal base capable of covering and sealing the lower-end opening from bottom, wherein the bottom seal base is provided with a matching portion for being combined with the matching port, the matching portion is concavely provided with a lower half compartment, and the lower half compartment and the upper half compartment are matched to form an inner space of the generation room;
an electrothermal device provided in the generation room and connected, via an electrical wire, to an electrical connection end having one end thereof exposed out of a bottom portion of the bottom seal base;
a drawing element having a drawing-in end penetrated into the oil compartment, an exchange end provided thereon located in the generation room, wherein the drawing element is combined with the electrothermal device in a manner enabling heat exchange.

2. The miniature gaseous substance release device of claim 1, wherein the matching port provided in the generation room and the matching portion are in a watertight combination.

3. The miniature gaseous substance release device of claim 1, wherein a crossover port is reserved between the air collector provided in the generation room and the bottom seal base for enabling the drawing element to overpass.

4. The miniature gaseous substance release device of claim 1, wherein the electrical connection end is provided with two relay terminals, and each of the relay terminals has one end thereof facing outwards and connected to an external power and an inner end thereof connected to the electrothermal device via a wire.

5. The miniature gaseous substance release device of claim 1, wherein atmospheric pressure is guided inwards into the oil compartment via a gap within assembly of the drawing element.

6. The miniature gaseous substance release device of claim 1, wherein atmospheric pressure is guided into the generation room via a balance channel provided on the bottom seal base.

7. The miniature gaseous substance release device of claim 1, wherein the top cover is provided with a supply hole for supplying an oil agent and capable of being opened and closed.

8. The miniature gaseous substance release device of claim 1, wherein the vertical trough is an upright trough which is communicated with the upper-end opening and the lower-end opening, and the top cover is sealingly combined with the bottom seal base along a longitudinal direction of the vertical trough.

9. The miniature gaseous substance release device of claim 1, wherein a joint portion is provided in a position where the relay terminals of the electrical connection end are exposed, and the joint portion is externally connected to a connection element for electrical transmission.

10. The miniature gaseous substance release device of claim 1, wherein the electrothermal device has a fitting space provided therein for enabling contacting and fitting over an outer surface of the exchange end provided on the drawing element.

11. The miniature gaseous substance release device of claim 1, wherein a thermal diffusion element is indirectly provided between the fitting space of the electrothermal device and the drawing element.

12. The miniature gaseous substance release device of claim 1, wherein the thermal diffusion element has an air slot provided on an inner surface thereof, or the air slot is provided with an air hole perpendicular thereto.

13. The miniature gaseous substance release device of claim 1, wherein the electrothermal device is an electrothermal coil, and a fitting space resulted from winding the electrothermal coil enables fitting over an outer surface of the drawing element.

14. The miniature gaseous substance release device of claim 1, wherein the electrothermal device and the electrical connection end are electrically connected via a passive switch.

15. The miniature gaseous substance release device of claim 14, wherein the passive switch is selected from either an element operated under the action of gas pressure, or an element operated by thermal sensitive effect.

16. The miniature gaseous substance release device of claim 1, wherein a release channel capable of isolating the oil compartment is provided between the connection tube of the air collector and the perforation of the top cover, an inner end of the release channel is a collection end for connecting to the generation room, and an outer end thereof is a release outlet coaxially combined with the perforation of the top cover in a watertight manner.
